(19) United States
(12) Patent Application Publication
(10) Pub. No.: US 2009/0130754 A1
(43) Pub. Date: May 21, 2009

(54) Method for culturing human embryonic stem cells

(75) Inventors: Hyung-Min Chung, Gyeonggi-do (KR); Soo-Hong Lee, Seoul (KR); Si-Nae Kim, Seoul (KR); Min-Jeong Kim, Incheon (KR)

Correspondence Address: THE NATH LAW GROUP, 112 South West Street, Alexandria, VA, 22314, US

(73) Assignees: CHABIOTECH CO., LTD., Seoul (KR); COLLEGE OF MEDICINE POCHON CHA UNIVERSITY INDUSTRY-ACADEMIC COOPERATION FOUNDATION, Gyeonggi-do (KR)

(21) Appl. No.: 12/227,793

(22) PCT Filed: May 31, 2007
(86) PCT No.: PCT/KR2007/002640

§ 371 Date: Nov. 26, 2008

(30) Foreign Application Priority Data
Jun. 1, 2006 (KR) 10-2006-0049546

(51) Int. Cl. C12N 5/06; C12N 5/08

(52) U.S. Cl. 435/357; 435/366

(57) ABSTRACT

The present invention relates to a method for culturing human embryonic stem cells (hESCs) in a hESC culture medium comprising a porous membrane, feeder cells being attached to a bottom of the porous membrane and a method for recovering human embryonic stem cells using the same.

### TECHNICAL FIELD

The present invention relates to a method for culturing human embryonic stem cells and a method for recovering human embryonic stem cells using the same.

### BACKGROUND ART

Human embryonic stem cells (hESCs) retain totipotency and can be differentiated into three germ cell layers (endodermal, ectodermal, mesodermal) which organize the human body. Thus, studies of hESCs can provide important clues for primitive aspects of early stages of human differentiation and can play a critical role in studies of cell therapy for diseases such as incurable diseases. In this respect, cell therapy using hESCs had received much attention. However, researchers had not succeeded in culturing hESCs due to its specificity although the success in culturing mouse embryonic stem cells had been reported.

In 1998, Thomson et al. reported the success in culturing hESCs (Thomson J A, Itskovitz-Eldor J, Shapiro S S, Waknitz M A, Swiergiel J J, Marshall V S, Jones J M, Embryonic stem cell lines derived from human blastocysts. Science (1998) 282: 1145-7). Since then, therapy studies using hESCs have faced a major turning point, and much attention has been paid to infinite potentialities of hESCs.

Although the success in culturing hESCs has been reported, some problems should be solved for clinical application. One of these problems is contamination of hESCs involved in cell culture. According to the most currently available hESC culture method which is developed based on the Thomson's method, hESCs are cultured in tissue culture media supplemented with leukemia inhibitory factor (LIF) using mouse embryonic fibroblasts (MEFs) as feeder cells. Unlike a method for culturing mouse embryonic stem cells, differentiation of hESCs cannot be prevented simply by addition of LIF. Thus, feeder cells capable of preventing rapid growth of hESCs are still absolutely required (Reubinoff B E, PEra M F, Fong C, Trounson A, Bongso A, Embryonic stem cell lines from human blastocysts: somatic differentiation in vitro. Nat Biotechnol. (2000) 18(4): 399-404).

Various techniques for culturing hESCs through new culture media conditions and co-culture of hESCs with animal stromal cells have been studied. However, it has been reported that clinical applications of these techniques might be still limited by problems caused by the use of animal cells and nutrient materials derived from animal cells, and in particular, possibility of remaining of animal cells in hESCs. Thus, in order to reduce contamination and infection by animal cells, a method for culturing hESCs using, as feeder cells, human cells such as human fibroblasts, human bone marrow stromal cells, and amniotic fluid cells, instead of using mouse fibroblasts has been developed. However, such a method cannot achieve a long-term serial subculture, and thus, it is difficult to secure large amounts of stem cells for cell therapy. Also, even though human cells are used as feeder cells, it is difficult to effectively isolate only cultured stem cells from the feeder cells. Thus, culture and isolation techniques suitable for clinical applications are absolutely required.

Hence, a technique for selectively isolating cultured hESCs without cell contamination and damage during and after culture is considered to be a very critical technique for realization of clinical application of hESCs.

Conventionally, an enzyme treatment technique and a mechanical technique are used to effectively isolate stem cells from feeder cells.

First, with respect to the enzyme treatment technique, large amounts of hESCs can be recovered for a short period by treating a solution containing an enzyme, such as collagenase, trypsin, or dispase, on culture dishes (Xu C, Inokuma M S, Denham J et al. Feeder free growth of undifferentiated human embryonic stem cells. Nat Biotechnol (2004), 19, 971-974; Richards M, Fong C Y, Chan W K et al. Human feeders support prolonged undifferentiated growth of human inner cell masses and embryonic stem cell. Nat Biotechnol (2002), 20, 933-936; Hovatta O, Mikkola M, Gertow K et al. A culture system using human foreskin fibroblasts as feeder cells allows production of human embryonic stem cells. Hum Reprod (2003), 18, 1404-1409). However, during enzyme treatment, hESCs may be contaminated or adversely affected (e.g., Karyotypic abnormalities).

On the other hand, the mechanical technique is a mechanical isolation technique that scratches only hESCs using a pipette (Heins N, Englund M C, Sjoblom C et al. Derivation, characterization, and differentiation of human embryonic stem cells. STEM CELLS (2004), 22, 367-376; Oh S K, Kim H S, Park Y B et al. Method for expansion of human embryonic stem cells, STEM CELLS (2005), 23, 605-609). The mechanical technique is a technique that manually isolates only hESCs using a thin pointed pipette while observing culture dishes through a microscope. This mechanical technique can exclude problems caused by enzyme treatment, but is laborious and time-consuming and feeder cells may be contained in recovered stem cells.

In order to solve these problems and to secure large amounts of hESCs, a combination technique was recently developed that boundaries between hESCs and feeder cells are mechanically isolated to some degrees and are then treated with enzymes (Oh S K, Kim H S, Park Y B et al. Method for expansion of human embryonic stem cells, STEM CELLS (2005), 23, 605-609). The combination technique can shorten the process duration, but is still laborious and time-consuming and exposure to enzymes is not solved.

In addition, a technique of isolating and recovering cultured hESCs using an automatic system can significantly shorten the isolation and recovery times (Alexis J, Christelle F-H, Kristine W et al. Automated Mechanical Passaging: A Novel and efficient method for human embryonic stem cell expansion, STEM CELLS (2006), 24, 230-235). However, the automatic technique may cause more serious cell contamination than the enzyme treatment technique and/or the mechanical technique due to accuracy defects of the automatic system.

As such, currently available techniques cannot solve the above-described fundamental problems. Even though large amounts of hESCs are acquired, the acquired hESCs are inappropriate to be clinically applied for human treatments.

### DISCLOSURE OF THE INVENTION

### Technical Problem

While searching for a method for culturing human embryonic stem cells (hESCs) which can be easily isolated while significantly reducing contamination by feeder cells, the present inventors have found that when culturing hESCs on a porous membrane having feeder cells attached to a bottom thereof, hESCs can be isolated easily and effectively and yielded with high purity.

### Technical Solution

Therefore, the present invention provides a method for culturing hESCs using a porous membrane.

The present invention also provides a method for recovering hESCs from a culture solution obtained by the culture method.

### BRIEF DESCRIPTION OF THE DRAWINGS

FIG. 1 is a graph illustrating degree of migration of feeder cells through pores of a porous membrane;

FIG. 2 is a graph illustrating the adhesion rate of human embryonic stem cells (hESCs) with respect to the concentration of feeder cells attached to a porous membrane; and

FIG. 3 is an optical microscopic image showing hESCs cultured on a porous membrane positioned on feeder cells and a staining image showing expression of undifferentiated cells.

### MODE FOR INVENTION

According to an aspect of the present invention, there is provided a method for culturing hESCs in a hESC culture medium comprising a porous membrane, feeder cells being attached to a bottom of the porous membrane.

According to another aspect of the present invention, there is provided a method for recovering hESCs, which comprises culturing hESCs using the above culture method and isolating the hESCs from the porous membrane.

Hereinafter, the present invention will be described in detail.

In accordance with a culture method of the present invention, human embryonic stem cells (hESCs) are cultured in a culture medium including a porous membrane, feeder cells being attached to a bottom of the porous membrane. Thus, interactions between the cultured hESCs and the feeder cells can be continuously maintained, and the cultured hESCs can be adhered and distributed on the porous membrane while maintaining an undifferentiated state. Therefore, after the culture is completed, it is possible to mechanically recover the cultured hESCs from the porous membrane without enzyme treatment, thereby solving problematic contamination that may be caused by enzyme treatment and the feeder cells.

The term “human embryonic stem cells (hESCs)” refers to pluripotent cells derived from the inner cell mass of human blastocysts. For example, hESCs may be CHA-hES3 (Ahn S E, Kim S, Park K H, Moon S H, Lee H J, Kim G J, Lee Y J, Park K H, Cha K Y, Chung H M. Primary bone-derived cells induce osteogenic differentiation without exogenous factors in human embryonic stem cells. Biochem Biophys Res Commun. (2006) 10; 340(2): 403-408) or the like, but the present invention is not limited thereto. In addition, hESCs can be easily established by those skilled in the art.

In the culture method of the present invention, a porous membrane attached with feeder cells is used. Through pores of the porous membrane, nutrients are supplied to hESCs, and hESCs can be maintained in an undifferentiated state. Here, the porous membrane is a membrane to which cells such as feeder cells can be adhered, and thus, a material for the porous membrane is not limited provided that it is a polymer having a porous property. The porous membrane that can be used in the culture method of the present invention may be made of a cell adhesive polymer, such as polyethylene terephthalate, polyethersulfone, polyvinylidene fluoride, cellulose, nylon, polyethylene, polypropylene, polycarbonate, polyurethane, polyacrylate, polycaprolactone, or a copolymer thereof. More preferably, the cell adhesive polymer may be polyethylene terephthalate. A commercially available BD Falcon™ (BD Bioscience, U.S.A.) manufactured to be adapted for the size of culture wells may also be used. The porous membrane may have a pore size of 0.1 to 3 μm, and more preferably 1 to 2.5 μm.

The feeder cells may be feeder cells commonly used as feeder cells for hESCs. For example, the feeder cells may be mouse fibroblasts, mouse embryonic fibroblasts, human embryonic fibroblasts, human bone marrow cells, adult epithelial cells, or the like. Among them, mouse embryonic fibroblasts are preferred since they can more stably maintain the undifferentiated state and growth of hESCs.

The attachment of the feeder cells to the porous membrane can be achieved by various methods. For example, the feeder cells may be attached to the porous membrane by adding the feeder cells and a feeder cell culture medium to the porous membrane and culturing the feeder cells for 12 to 48 hours, preferably about 24 hours. The feeder cell culture medium may vary according to the feeder cells used, but may be appropriately selected by those skilled in the art considering known feeder cells and culture methods thereof. For example, when using STO cells as the feeder cells, the feeder cell culture medium may be Dulbecco's modified Eagles medium (DMEM) supplemented with fetal bovine serum (FBS), mercaptoethanol, and a nonessential amino acid. The porous membrane attached with the feeder cells may be washed with a physiologically compatible buffer solution, e.g., phosphate-buffered saline, to remove unnecessary materials such as FBS. The density of the feeder cells attached to the porous membrane may be 1.0×10⁵ to 5.0×10⁵ cells/well, and more preferably about 2.5×10⁵ cells/well.

The porous membrane prepared as described above is inserted into a hESC culture medium such that a feeder cell attachment surface of the porous membrane faces down.

The hESC culture medium may be selected from all known hESC culture media. For example, the hESC culture medium may be knockout DMEM (KO-DMEM) supplemented with serum replacement (SR), mercaptoethanol, nonessential amino acid, and bFGF.

In the culture method of the present invention, when needed, the porous membrane may be coated with various natural or synthetic materials, e.g., collagen, fibronectin, laminin, or Metrigel. For example, feeder cells may be attached to a porous membrane coated with collagen, fibronectin, or laminin. By doing so, the culture of hESCs can be effectively promoted by the above-described materials in the presence of the feeder cells.

The present invention also provides a method for recovering hESCs, which includes: culturing hESCs using the above-described culture method; and isolating hESCs from the porous membrane.

The isolation of hESCs from the porous membrane may be performed by a mechanical isolation method, for example, by scratching hESCs from the porous membrane using a pipette or the like. That is, the use of a mechanical isolation method can exclude an enzyme treatment, thus preventing problematic contamination that may be caused by enzymes. In particular, the porous membrane has a strength sufficient to mechanically scratch cultured cells, thus ensuring simple recovery of hESCs.

The hESCs recovered as described above have a normal karyotype and characteristics of undifferentiated cells. That is, the hESCs recovered as described above normally express Oct-4 and keep hESC morphologies intact as determined by immunochemical staining, RT-PCR, or the like. In addition, the hESCs express hESC markers, i.e., APL (alkaline phosphatase), SSEA (stage specific embryo antigen), TRA, etc., and have no trouble in embryoid body formation.

Hereinafter, the present invention will be described more specifically with reference to the following examples. The following examples are only for illustrative purposes and are not intended to limit the scope of the invention.

### EXAMPLE 1

Porous membranes (BD Falcon™, BD Bioscience, U.S.A.) having a pore size of 1 μm were placed in 6-well culture dishes. Then, mouse embryonic fibroblasts (STO cells, 2.5×10⁵ cells/well), which had been treated with mitomycin-C for two hours to prevent cell proliferation, and feeder cell culture media (90% DMEM supplemented with 10% FBS, 0.1 mM mercaptoethanol, and 1% nonessential amino acid (Gibco)) were added into the culture dishes, and the STO cells were cultured for 24 hours. The membranes attached with the STO cells were removed, washed twice with phosphate-buffered saline, and sufficiently immersed in hESC culture media (80% KO-DMEM supplemented with 20% SR, 0.1 mM mercaptoethanol, 1% nonessential amino acid (Gibco), and 4 ng/ml bFGF) such that the STO cells faced down. The clumps of hESCs (CHA-hES3) were finely split, and about 30 splits were seeded on the hESC culture media. At 48 hours after the seeding, it was determined whether or not hESCs were efficiently attached to the membranes. Then, the hESC culture media were replaced with new ones every day for five days. The hESC clumps grown in this manner were finely split, seeded on newly prepared feeder cell-attached porous membranes, and then cultured in the same manner as described above.

### EXAMPLE 2

hESCs were cultured in the same manner as in Example 1 except that porous membranes (BD Falcon™, BD Bioscience, U.S.A.) having a pore size of 1 μm which had been coated with fibronectin, collagen, laminin, and Metrigel, respectively, were placed in culture dishes.

### EXAMPLE 3

The hESCs cultured on the porous membranes in the culture media of Example 1 were scratched using a pipette without enzyme treatment to recover the hESCs.

### COMPARATIVE EXAMPLE 1

Migration of the STO cells used as feeder cells in BD Falcon™ (BD Bioscience, U.S.A.) inserts having pore sizes of 3 μm and 8 μm, respectively, was observed. As illustrated in FIG. 1, the porous membranes having pore sizes of 3 μm and 8 μm may cause contamination of hESCs due to migration of the STO cells, but the porous membranes having a pore size of 1 μm did not undergo the migration of the STO cells.

### COMPARATIVE EXAMPLE 2

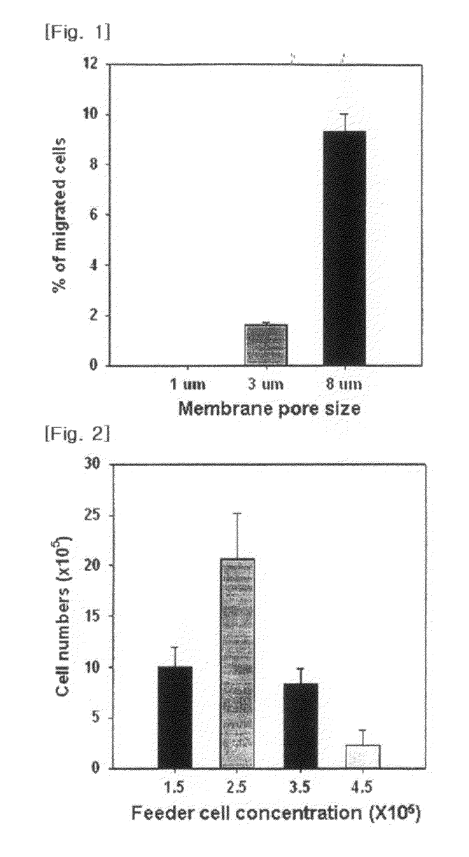

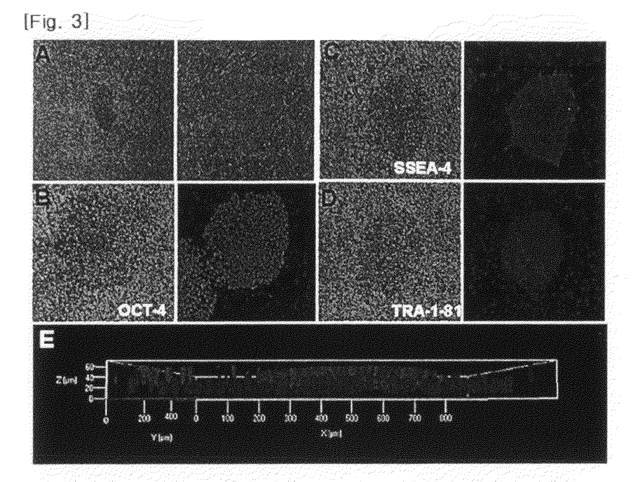

hESCs were cultured in the same manner as in Example 1 except that the concentrations of the STO cells used as feeder cells was adjusted to 1.5×10⁵, 3.5×10⁵, and 4.5×10⁵ cells/well, respectively. As illustrated in FIG. 2, when the concentration of STO cells is 2.5×10⁵ cells/well, the adhesion rate of hESCs onto porous membranes is the highest.

### COMPARATIVE EXAMPLE 3

Mouse embryonic fibroblasts (STO cells, 2.5×10⁵ cells/well), which had been treated with mitomycin-C for two hours to prevent cell proliferation, were added in 6-well culture dishes. Feeder cell culture media (90% DMEM supplemented with 10% FBS, 0.1 mM mercaptoethanol, and 1% nonessential amino acid (Gibco)) were added into the culture dishes, and the STO cells were cultured for 24 hours. The 6-well culture dishes were washed twice with phosphate-buffered saline to completely remove FBS. Then, hESC culture media (80% KO-DMEM supplemented with 20% SR, 0.1 mM mercaptoethanol, 1% nonessential amino acid (Gibco), and 4 ng/ml bFGF) were added to the 6-well culture dishes, and porous membranes (BD Falcon™, BD Bioscience, U.S.A.) having a pore size of 1 μm were added and immobilized in the 6-well culture dishes. The clumps of hESCs (CHA-hES3) were finely split, and about 30 splits were seeded on the hESC culture media. At 48 hours after the seeding, it was determined whether or not hESCs were efficiently attached to the membranes. As a result, adhesion of hESCs onto the membranes hardly occurred.

### COMPARATIVE EXAMPLE 4

Culture media of 80% KO-DMEM supplemented with 20% SR, 0.1 mM mercaptoethanol, 1% nonessential amino acid (Gibco), and 4 ng/ml bFGF were added in 6-well culture dishes, and porous membranes (BD Falcon™, BD Bioscience, U.S.A.) having a pore size of 1 μm were added and immobilized in the 6-well culture dishes. The clumps of hESCs (CHA-hES3) were finely split, and about 30 splits were seeded on the hESC culture media. At 48 hours after the seeding, it was determined whether or not hESCs were efficiently attached to the membranes. As a result, adhesion of hESCs onto the membranes did not occur.

As can be seen from Comparative Examples 3 and 4, if no feeder cells are attached to a bottom of a porous membrane, hESCs are not adhered to the porous membrane. Furthermore, feeder cells positioned in a culture dish does not significantly affect the adhesion of hESCs onto the porous membrane. That is, these results show that feeder cells positioned below a porous membrane play an important role in adhesion and culture of hESCs through pores of the porous membrane.

### EXPERIMENTAL EXAMPLE 1

In order to determine whether or not hESCs are in an undifferentiated state, the hESCs immobilized on the porous membranes in the culture media of Example 1 were fixed with 4% paraformaldehyde for 20 minutes, permeabilized with 0.1% Triton X-100 for five minutes, and treated with 1% normal goat serum at room temperature. Then, the hESCs were incubated with human specific antibodies, i.e., Oct4 (1:100), SSEA-4 (1:100), and Tra-1-81 (1:100) antibodies, at 4° C. for 12 hours. Cell samples were washed and incubated with rhodamine-conjugated goat anti-mouse IgG (1:800) secondary antibodies at room temperature for one hour in order to detect the primary antibodies. Stained samples were again washed and incubated with DAPI (1:5000) at room temperature for five minutes to stain cell nuclei. Images were analyzed with a fluorescence microscope (ApoTome, Carl Zeiss, Jena, Germany). FIG. 3 is an image showing hESCs after three-day culture on porous membranes having feeder cells attached thereto. In FIG. 3, (B) is an image showing hESCs co-stained with an undifferentiation marker Oct4 (red) and a nuclear stain DAPI (blue), (C) is an image showing hESCs co-stained with an undifferentiation marker SSEA-4 (red) and DAPI (blue), and (D) is an image showing hESCs co-stained with an undifferentiation marker Tra-1-81 (red) and DAPI (blue). (E) of FIG. 3 is an image showing a three dimensional structure derived from the image of (B) of FIG. 3 using a fluorescence microscope.

### INDUSTRIAL APPLICABILITY

According to a culture method of the present invention, human embryonic stem cells (hESCs) are cultured in a culture medium including a porous membrane, feeder cells being attached to a bottom surface of the porous membrane. Thus, interactions between the cultured hESCs and the feeder cells can be continuously maintained, and the cultured hESCs can be adhered and distributed on the porous membrane while maintaining an undifferentiated state. Therefore, after the culture is completed, it is possible to recover the cultured hESCs from the porous membrane without enzyme treatment, thereby solving problematic contamination that may be caused by enzyme treatment and the feeder cells. In addition, the culture method of the present invention is much less labor- and time-intensive than conventional hESC culture and isolation methods, and ensures stable and large-scale production of hESCs.

What is claimed is:

1. A method for culturing human embryonic stem cells (hESCs) in a hESC culture medium comprising a porous membrane, feeder cells being attached to a bottom of the porous membrane.

2. The method of claim 1, wherein the porous membrane is made of a cell adhesive polymer selected from the group consisting of polyethylene terephthalate, polyethersulfone, polyvinylidene fluoride, cellulose, nylon, polyethylene, polypropylene, polycarbonate, polyurethane, polyacrylate, polycaprolactone, and a copolymer thereof.

3. The method of claim 1, wherein the porous membrane has a pore size of 0.1 to 3 μm.

4. The method of claim 1, wherein the feeder cells are mouse fibroblasts, mouse embryonic fibroblasts, human embryonic fibroblasts, human bone marrow cells, or adult epithelial cells.

5. The method of claim 1, wherein the porous membrane is coated with collagen, fibronectin, laminin, or Metrigel.

6. A method for recovering hESCs, comprising culturing hESCs using the method of any one of claims 1 through 5; and isolating hESCs from the porous membrane.

7. The method of claim 6, wherein the isolating hESCs from the porous membrane is carried out by scratching hESCs from the porous membrane.
